(19) United States
(12) Patent Application Publication
(10) Pub. No.: US 2026/0023230 A1
(43) Pub. Date: Jan. 22, 2026

(54) SYMMETRICAL DIRECTIONAL COUPLER BASED ON DIELECTRIC WAVEGUIDES

(71) Applicant: Siemens Healthineers AG, Forchheim (DE)

(72) Inventor: Andreas FACKELMEIER, Thalmaessing (DE)

(73) Assignee: Siemens Healthineers AG, Forchheim (DE)

(21) Appl. No.: 19/267,710

(22) Filed: Jul. 14, 2025

(30) Foreign Application Priority Data
Jul. 17, 2024 (EP) 24189133.2

(51) Int. Cl. G02B 6/43; A61B 5/00; A61B 5/055; A61B 6/00; A61B 6/03; A61B 6/04

(52) U.S. Cl. CPC G02B 6/43; A61B 5/0013; A61B 5/0017; A61B 5/055; A61B 5/704; A61B 6/032; A61B 6/0407; A61B 6/56

(57) ABSTRACT

A data transmission apparatus is provided which has a first dielectric waveguide with a first coupling section and a second dielectric waveguide which can be moved in a direction of movement opposite the first coupling section and has a first variable distance to the first coupling section. Furthermore, the data transmission apparatus has a third dielectric waveguide which has a second coupling section and a second variable distance to the second dielectric waveguide. The first dielectric waveguide is fixed with respect to the third dielectric waveguide. The first coupling section and the second coupling section are of equal length in the direction of movement and lie opposite one another perpendicular to the direction of movement.

### CROSS-REFERENCE TO RELATED APPLICATION(S)

The present application claims priority under 35 U.S.C. § 119 to European Patent Application No. 24189133.2, filed Jul. 17, 2024, the entire contents of which is incorporated herein by reference.

### FIELD

One or more example embodiments relates to a data transmission apparatus for contactless data transmission, having a first, second and third dielectric waveguide. The first dielectric waveguide has a first coupling section and the third dielectric waveguide has a second coupling section. The second dielectric waveguide can be moved opposite the first coupling section and the second coupling section.

Furthermore, one or more example embodiments relates to an imaging apparatus and a method for contactless data transmission.

Independent of the grammatical term usage, individuals with male, female or other gender identities are included within the term.

### RELATED ART

A computed tomograph typically has a fixed base and a so-called gantry, the gantry moving relative to the fixed base during operation, i.e., during data acquisition via an X-ray detector, and performing a rotational movement. The X-ray detector, with the help of which data is generated for image creation, is usually part of the gantry, so that the data generated during operation must be transmitted from the gantry via a data transmission path to the fixed base and usually to a processing unit for the generated data.

The transmission of the generated data via the data transmission path typically takes place without temporary storage or relevant buffering and accordingly quasi parallel to the generation of corresponding data and therefore also during the rotation of the gantry. Data transmission paths suitable for this are known in principle from the prior art, in most cases a so-called slip ring being part of a corresponding data transmission path.

An embodiment of a data transmission path suitable for such data transmission is described in DE 10 2016 208 539 A1. This comprises a dielectric waveguide and is also suitable for contactless data transmission.

The ever-increasing data volumes resulting from the higher resolution in imaging and the resulting requirements for the data rate during transmission represent an as yet unresolved problem. One approach is to improve data rates by using directional couplers which are based on dielectric waveguides. Transmission is essentially achieved by electromagnetic, in particular optical, coupling of a signal which is conducted through a dielectric waveguide on the gantry into another dielectric waveguide on the holding frame via the air gap.

A serious problem here is an axially and radially varying offset of the holding frame to the gantry during operation of the CT as a result of the rotation of the gantry. Due to this varying offset, the distance between the two dielectric waveguides, which are the main components of the directional coupler, is not constant. Viewed from the respective plane of the two waveguides, such a variation in distance has a normal and a longitudinal component, the variation of the longitudinal component having a considerable effect on the coupling strength, i.e. on the received signal strength. The coupling strength demonstrates a complex functional interrelationship between the distance of the two waveguides to one another and their respective geometric parameters (primarily: coupling length and cross-section).

Consideration was given to inserting a 60 GHZ amplifier on the transmit or TX side or better as an LNA (low noise amplifier) on the receive or RX side, which would result in very high additional costs. Furthermore, optimizing the entire transmission path for lower maximum RF attenuation, low attenuation variation (dynamics) and high mechanical stability is very complex, which would also lead to very high costs.

### SUMMARY

However, the problem mentioned is not only limited to the field of medical tomographs but is generally relevant if data or energy is to be transmitted from one device part to another in a device with two device parts which perform a movement relative to one another during the operation of the device, and this could not, or could not reasonably, be achieved via a cable connection on account of the relative movement.

One or more example embodiments provides an advantageous solution for data transmission.

This is achieved by a data transmission apparatus as well as by an imaging apparatus and a method according to the independent claims. Preferred developments are included in the retrospective claims. The advantages and preferred embodiments cited with regard to the data transmission apparatus can also be applied analogously to the imaging apparatus and the method, and vice versa.

### BRIEF DESCRIPTION OF THE DRAWINGS

Exemplary embodiments of the invention are explained in more detail hereinafter with reference to diagrammatic drawings. The diagrams show:

FIG. 1A diagrammatic view of a computed tomograph;

FIG. 2 An exemplary embodiment of a coupler;

FIG. 3 Another exemplary embodiment of a coupler;

FIG. 4 Yet another exemplary embodiment of a coupler;

FIG. 5A sectional view of the coupler in FIGS. 2 to 4 in a central position;

FIG. 6 The view of FIG. 5 with an axial offset of the second waveguide;

FIG. 7 The view of FIG. 5 with a radial offset of the second waveguide; and

FIG. 8 An alternative arrangement of the waveguide of the coupler.

### DETAILED DESCRIPTION

According to one or more example embodiments, a data transmission apparatus for contactless data transmission is thus provided. Data transmission preferably takes place in the optical wavelength range (e.g. at 60 GHZ). Therefore, the data transmission apparatus is realized with dielectric waveguides.

The data transmission apparatus has a first dielectric waveguide with a first coupling section. The first coupling section is the section which is primarily used for coupling or data transmission.

Furthermore, the data transmission apparatus has a second dielectric waveguide which can be moved opposite the first coupling section in a direction of movement. This means that the second dielectric waveguide can be moved opposite the first dielectric waveguide. The spatial proximity between the first coupling section and the second dielectric waveguide results in a coupling which enables contactless data transmission. The first coupling section preferably runs in the longitudinal direction of the second dielectric waveguide. However, this also means that in this case the first and second dielectric waveguides run parallel to one another.

The first coupling section has a first variable distance in relation to the second dielectric waveguide. This variable distance usually results from the movement of the second dielectric waveguide in relation to the first dielectric waveguide. Due to mechanical tolerances of the components involved, typically a variation in distance is also produced perpendicular to the direction of movement in which the two waveguides move relative to one another.

The data transmission apparatus also has a third dielectric waveguide which has a second coupling section and a second variable distance to the second dielectric waveguide. There is therefore also a direct coupling between the third dielectric waveguide and the second dielectric waveguide. For example, the third dielectric waveguide is arranged opposite the first dielectric waveguide in relation to a perpendicular to the direction of movement.

The first dielectric waveguide is fixed in relation to the third dielectric waveguide. As the second dielectric waveguide moves relative to the first dielectric waveguide, this also results in the second dielectric waveguide moving relative to the third dielectric waveguide. This enables contactless data transmission from the moving second dielectric waveguide to both the fixed first dielectric waveguide and the fixed third dielectric waveguide.

The first coupling section and the second coupling section are of equal length in the direction of movement and are arranged opposite one another (preferably in parallel) perpendicular to the direction of movement. The equal length of the coupling sections has the advantage that the first and third dielectric waveguides can in principle be the same shape, which can result in cost savings. Furthermore, they are opposite one another perpendicular to the direction of movement, as a result of which it may be possible to save space, in particular if the three dielectric waveguides run parallel to one another in the coupling area, i.e. over the length of the coupling sections. If the coupling sections have the same dimensions and are positioned symmetrically to the second dielectric waveguide, this results in the same dynamic range for both couplings respectively, so that the subsequent signal processing can also be the same and therefore more cost-effective.

According to an exemplary embodiment, it is provided that in relation to data transmission by optical coupling either the second dielectric waveguide is assigned to a transmitting unit of the data transmission apparatus and the first waveguide together with the third dielectric waveguide is assigned to a common receiving unit of the data transmission apparatus, or the second dielectric waveguide is assigned to a receiving unit of the data transmission apparatus and the first and/or third dielectric waveguide is assigned to a transmitting unit of the data transmission apparatus. These two variants refer, for example, to the case where data needs to be transmitted from a rotating gantry to the outside or vice versa to the inside of the rotating gantry. Of course, the respective transmit and receive function can also change if corresponding transceiver units are used. The change can also take place at very short intervals under certain circumstances.

For coupling, it is generally necessary that the coupled waveguides have a certain proximity to one another. This is the only way to ensure that the coupling strength does not fall below a critical value. A maximum distance in this regard also depends, inter alia, on the wavelength of the electromagnetic signals to be transmitted.

In a further exemplary embodiment, the first coupling section and the second coupling section are arranged symmetrically to one another perpendicular to the direction of movement. This means that their coupling to one another is equal if they are each at the same distance from the moving second dielectric waveguide. If necessary, all sections of the first dielectric waveguide can also be symmetrical in relation to the corresponding sections of the third dielectric waveguide. In this case, the two waveguides can therefore be the same shape, resulting in manufacturing advantages.

In a special embodiment, the first coupling section and the second coupling section are arranged parallel to the second dielectric waveguide. This means that there is uniform coupling between the first and the second waveguide, but also between the third and the second waveguide over the length of the coupling sections.

In a preferred exemplary embodiment, it is provided that the second dielectric waveguide passes between the first and the third dielectric waveguide. In particular, the second dielectric waveguide passes between the first and the second coupling sections. Therefore, for example, if the first and second coupling sections together form the boundaries of a common sheath, the second dielectric waveguide passes through the volume bounded by the sheath. The mutual variation in distance then results in the constellation that the first variable distance between the first and the second waveguide increases when the second variable distance between the third and the second waveguide decreases and vice versa.

According to a further exemplary embodiment, it is provided that the second dielectric waveguide is formed on a circular path with a circle center and a path radius, and the coupling sections are arranged in the same position in the circumferential direction as follows:
a) one of the two coupling sections radially outside the circular path and the other coupling section radially inside the circular path,
b) both coupling sections with the path radius away from the circle center axially on both sides of the circular path,
c) both coupling sections radially inside the circular path and axially offset from it on both sides, or
d) both coupling sections radially outside the circular path and axially offset from it on both sides.

The second dielectric waveguide is therefore located on a circular path which can be realized on a rotating, round unit, such as a gantry. Both coupling sections are located in the same position in the circumferential direction. This means that the coupling sections both couple into and out of the same section of the second dielectric waveguide. Furthermore, in the case of decoupling from the second dielectric waveguide, this means that the decoupled signals in the first and third waveguides or in the first and second coupling sections have the same phase. In variant a), the two coupling sections face one another radially and the second dielectric waveguide runs between them. This can be used to counteract a radial imbalance in a gantry, for example.

In variant b), the two coupling sections are axially opposite one another and the second dielectric waveguide runs between them. In this case, axial non-uniformity can be counteracted during signal evaluation.

In variant c), the two coupling sections are axially opposite one another within the circular path. The second dielectric waveguide runs, axially slightly offset, radially across both coupling sections. With this coupling constellation, axial imbalances of the second dielectric waveguide can also be compensated. However, it should be noted that the distance (in relation to the waveguide centers) from the second waveguide to the first coupling section changes differently in terms of amount to the distance from the second waveguide to the second coupling section.

Variant d) corresponds in principle to variant c), the two coupling sections only being arranged radially outside the circular path. The coupling properties are therefore essentially the same for both variants.

According to a further exemplary embodiment, a respective receiver for detecting electromagnetic signals in the respective waveguide is arranged on the first and third waveguides, and signals from the two receivers are linked to one another. This means that although two receivers are provided, their signals are linked, preferably added together. Depending on the distance between the second dielectric waveguide and the first or second coupling section, different coupling strengths and therefore different signals result at the two receivers. This means that there are always two signals of different strengths available, the larger of which can be utilized.

According to a further exemplary embodiment, a single receiver is arranged on the first and the third dielectric waveguide, which receives electromagnetic signals coupled from the second dielectric waveguide into both the first and the third dielectric waveguides. This therefore means that the first and the third waveguides are combined in the wave direction after coupling with the second waveguide and an overall signal is obtained or evaluated with a single receiver. In this manner, one receiver can therefore be saved.

In a specific embodiment, it is provided that the first and the third dielectric waveguides in one section form the second dielectric coupler with which an electromagnetic signal can be transmitted from the third to the first dielectric waveguide, and the single receiver is arranged directly on the first dielectric waveguide in order to also receive the electromagnetic signal coupled from the third into the first dielectric waveguide. Only the first dielectric waveguide is therefore equipped with a receiver. This receives the electromagnetic signal coupled from the second dielectric waveguide into the first dielectric waveguide directly. The electromagnetic signal coupled into the third dielectric waveguide is first conducted to the second coupler and there coupled into the first dielectric waveguide before it can be detected by the single receiver. No receiver is therefore necessary on the third dielectric waveguide.

In a particularly preferred embodiment, the coupling section of the first dielectric waveguide is spaced a first distance from the second coupler, the coupling section of the third dielectric waveguide is spaced a second distance from the second coupler, and the second distance corresponds to a sum of the first distance and a multiple of a predetermined wavelength in the dielectric waveguides for data transmission. As a consequence, the two electromagnetic signals are synchronized on arrival at the second coupler because the third dielectric waveguide is longer than the first waveguide by a multiple (including 0 and 1) of the wavelength intended for data transmission, as viewed from the end of the (first) coupler to the beginning or center of the second coupler.

In an alternative embodiment, it is provided that the first and third dielectric waveguide are guided to a dielectric combiner or in one section form the dielectric combiner with which an electromagnetic signal can be conducted from both the first and the third dielectric waveguide into the single receiver. The first and the third dielectric waveguides are therefore combined or merged into a single waveguide and the single receiver then registers electromagnetic signals in the combined waveguide section. In the event of the reversal of data transmission, the combiner can also be seen as a divider.

According to one or more example embodiments, an imaging apparatus is also provided which has a data transmission unit, as described above. This enables an alternative or simplified imaging apparatus to be configured compared to the prior art.

A specific exemplary embodiment provides that the second dielectric waveguide is part of a rotatable gantry with a gantry support structure and the first and the third dielectric waveguides are part of a fixed base (in the present document also referred to as a holding frame). The second dielectric waveguide can be fixed to the gantry support structure as part of the transmitting unit. This enables data to be reliably transmitted from the gantry to the fixed base with a high bandwidth and vice versa.

The imaging apparatus can be designed as a computed tomograph or a magnetic resonance tomograph. Both tomographs have corresponding gantries and therefore the need to transmit data from the gantry to the outside at high speed.

The above object is also achieved according to one or more example embodiments by a method for (optical) contactless data transmission, comprising
movement of a second dielectric waveguide during data transmission relative to a first coupling section of a first dielectric waveguide in a direction of movement, (e.g. in a longitudinal direction of the second dielectric waveguide), a first distance of the second dielectric waveguide (e.g. lateral surface to opposite lateral surface or center line to center line; other than zero) to the first coupling section varying, and a second distance of a second coupling section of a third dielectric waveguide (opposite the first dielectric waveguide and) to the second dielectric waveguide varying,
the first dielectric waveguide being fixed in relation to the third dielectric waveguide,
the first coupling section and the second coupling section being of equal length in the direction of movement and being opposite one another perpendicular to the direction of movement.

With regard to the method, the same advantages and development options arise analogously as for the above imaging apparatus or the above data transmission unit.

An electromagnetic signal is therefore transmitted via optical directional coupling between the second dielectric waveguide on the one hand and the first or third dielectric waveguide on the other hand. Optical directional coupling is to be understood here as coupling in which an optical signal conducted through a waveguide, the modes of which propagate partially outside the waveguide, “crosstalk” into another waveguide at least partially parallel to the waveguide, hereby also providing a propagating optical signal there.

The data transmission apparatus is preferably used in an imaging apparatus, as it is described in particular in DE 10 2016 208 539 A1, or in DE 10 2015 223 068 A1. The data transmission apparatus described here then replaces the data transmission units or transmission paths described in these publications. Reference is therefore expressly made to the descriptions in both publications at this point.

The exemplary embodiments described in more detail hereinafter represent preferred embodiments of the present invention.

Corresponding parts are each marked with the same reference characters in all the figures.

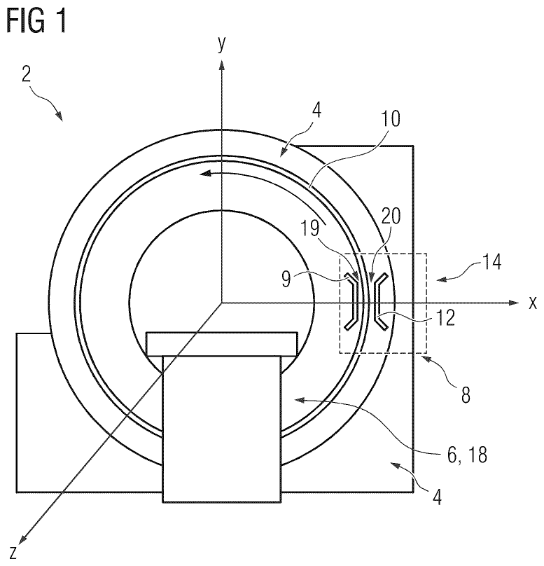

An imaging apparatus described hereinafter by way of example and sketched in FIG. 1 is embodied as a computed tomograph 2 and has a fixed base 4 as well as a rotatable gantry 6. Here, the gantry 6 is designed according to a known principle for generating image data via an X-ray detector (not shown) and accordingly, during operation of the computed tomograph 2, data is then generated which is to be transmitted from the rotatable gantry 6 to the fixed base 4 during the rotation of the gantry 6 and is transmitted via a data transmission unit 8.

A first dielectric waveguide 9, a second dielectric waveguide 10 and a third dielectric waveguide 12 are part of this data transmission unit 8. The first and third dielectric waveguides 9 and 12 are attached to the base 4, while the second dielectric waveguide 10 is attached to the gantry 6. The dielectric waveguides 9, 10 and 12 are arranged relative to one another in such a manner that there is an air gap 19 between the first dielectric waveguide 9 and the second dielectric waveguide 10 and an air gap 20 between the second dielectric waveguide 10 and the third dielectric waveguide 12. The respective air gap 19 or 20 can be approximately 1 mm on average and may vary in a range between 0.5 mm and 1.5 mm when the gantry 6 is rotated.

Image data can be transmitted across this air gap 20 from the second dielectric waveguide 10 to the first and third dielectric waveguides 9 and 12 and thus from the rotatable gantry 6 to the fixed base 4 according to a principle known per se. The second dielectric waveguide 10 is thus part of a transmitting unit, while the first and third dielectric waveguides 9, 12 are part of a receiving unit. The second dielectric waveguide 10 and the first and third dielectric waveguides 9, 12 thus form a coupler 14 which couples the base 4 and the gantry 6 in terms of signal technology.

In a preferred embodiment, the second dielectric waveguide 10 is annular in design or assumes an annular shape in the assembled state, as indicated in FIG. 1. The second dielectric waveguide 10 thus runs around the circumference of the rotatable gantry 6 in the assembled state and is preferably arranged recessed in a groove. This groove 16 is part of a gantry support structure 18 and preferably forms a second part of a plug connection, via which the second dielectric waveguide 10 is attached to the gantry support structure 18.

The first dielectric waveguide 9 and the third dielectric waveguide 12 have an approximately C-shaped design in the axial view in the present example. Both waveguides 9 and 12 are arranged here in mirror symmetry to the second dielectric waveguide 10. The first dielectric waveguide 9 is located radially inside the second dielectric waveguide 10 and the third dielectric waveguide 12 is located radially outside. The open side of the C-shape of the first dielectric waveguide 9 points to the center of the gantry 6, while the open side of the third dielectric waveguide 12 is directed radially outwards.

In an alternative embodiment, the first to third dielectric waveguides 9, 10, 12 are located in the same radial position. In an axial direction, the second dielectric waveguide 10 is located between the first and third dielectric waveguides 9, 12. The air gaps or distances between the waveguides could be of the same order of magnitude as in the preceding exemplary embodiment.

In a mixed form (cf. FIG. 8), the first and third dielectric waveguides 9, 12 are in the same radial position and the second dielectric waveguide 10 is radially below or above them. Here, the distance between two waveguides could refer to the distance between the centers of two waveguides. The coupling strength could then be regarded as a function of these distances.

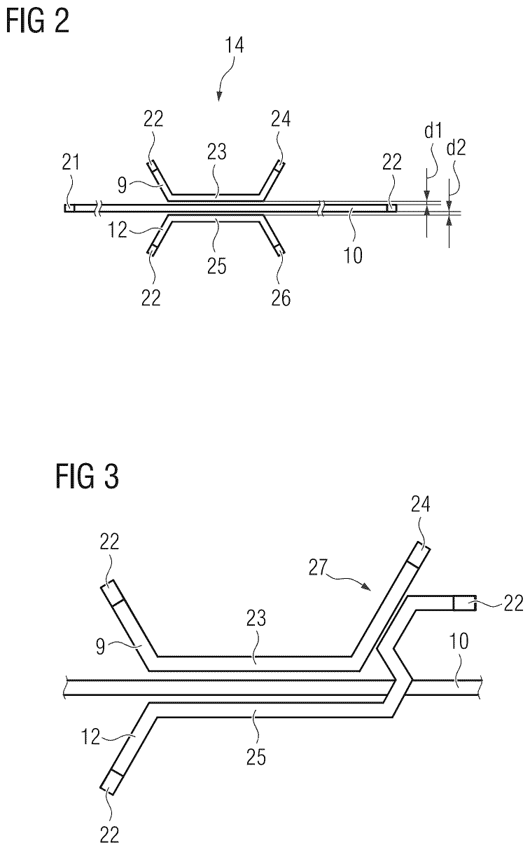

In FIG. 2, the coupler 14 is shown enlarged in accordance with the exemplary embodiment of FIG. 1. The second dielectric waveguide 10 is shown here diagrammatically as a straight line without a gantry 6. However, this only corresponds to a simplified representation, in particular if the second dielectric waveguide 10 is annular in design and the central axis is arranged perpendicular to the image plane. Alternatively, the central axis (axis of rotation of the gantry 6) can also extend parallel to the image plane, so that the annular second dielectric waveguide 10 actually represents a straight line in the plan view. A transmitter 21 is provided at one end of the second dielectric waveguide 10, which can also have reception functionality. At the other end of the second dielectric waveguide 10 there is, for example, a termination 22.

The first waveguide 9 and the third waveguide 12 are arranged opposite one another on both sides of the second dielectric waveguide 10. The first dielectric waveguide 9 has, for example, a first coupling section 23 in its central area. In its end sections 24, it can be guided away from the second dielectric waveguide 10, resulting in the C-shape of the first dielectric waveguide 9 shown diagrammatically in FIG. 2. A termination 22 can be arranged at one end and a first receiver 24 at the other end. The transmitter 1 emits light through the second dielectric waveguide 10, which is coupled into the first dielectric waveguide 9 and guided therein to the first receiver 24. The first receiver 24 is therefore located downstream of the first coupling section 23 in the direction of the beam.

The third waveguide 12 is shaped and arranged axially symmetrically to the first dielectric waveguide 9. The second dielectric waveguide 10 symbolizes the mirror axis. Accordingly, the third dielectric waveguide 12 has a second coupling section 25 in its central area and a termination 22 at one end as well as a second receiver 26 at the other end.

The first coupling section 23 and the second coupling section 25 are preferably arranged parallel to one another and the second dielectric waveguide 10 runs between them. The first coupling section 23 and the second dielectric waveguide 10 are at a distance d1 from one another. These distances d1 and d2 vary with the eccentricity or imbalance of the gantry 6 or the second dielectric waveguide 10 assembled thereon. This imbalance occurs when the axis of rotation of the gantry 6 does not correspond to one of its main axes of inertia. However, the sum of the distances d1+d2 is constant.

The coupling strength between the second dielectric waveguide 10 and the first dielectric waveguide 9 or the third dielectric waveguide 12 depends on the respective distance d1 or d2. The strength of the coupling between the respective dielectric waveguide pairs corresponds to a complex, non-monotonous function as a function of the respective distance d1 or d2. The physical interaction processes relevant for the respective coupling have a functional distance dependence which does not necessarily result in a monotonous increase in the coupling strength with decreasing distance, but in the aforementioned complex functional dependence, for example as a result of destructive interference of wave components of the signal.

To reduce this dependence, the light of the rotating second dielectric waveguide 10 is coupled on both sides into the respective slightly spaced coupling section 23, 25 or the dielectric waveguides 9, 12. The signals of the two receivers 24 and 26 can be added together, for example, as a result of which the distance dependence of the coupling strength is reduced.

The first dielectric waveguide 9 and the third dielectric waveguide 12 or their coupling sections 23 and 25 are fixed in relation to one another. This means that the sum of the distances dl and d2 is constant as the width of the second dielectric waveguide 10 can be regarded as uniform over its length. Therefore, if the distance dl decreases at a point in time when the gantry 6 is imbalanced, the distance d2 increases and vice versa. However, this does not mean that the sum of the coupling strengths is constant. Rather, as explained above, relevant non-linearities determine the (total) coupling strength.

FIG. 3 shows an alternative embodiment in which it is possible to dispense with the second receiver 26 compared with the previous embodiment. The first waveguide 9 has essentially the same shape as in the exemplary embodiment of FIG. 2. At one end, it has the termination 22 and at the other end the first receiver 24. The second coupling section 25 of the third waveguide 12 here likewise runs parallel to the first coupling section 23 of the first dielectric waveguide 9. The second dielectric waveguide 10 also moves through the two parallel coupling sections 23 and 25.

Unlike in the example of FIG. 2, the first and the third dielectric waveguides 9 and 12 also have a common dielectric coupling section 27. There, the first and the third dielectric waveguides 9 and 12 run parallel for a distance without the second dielectric waveguide 10 passing between them. In the common dielectric coupling section 27, direct optical coupling takes place between the two waveguides 9 and 12. This means that the light which is fed into the second dielectric waveguide 10 and coupled into the third dielectric waveguide in the coupling section 25 is coupled into the first dielectric waveguide 9 in the common dielectric coupling section 27. At the same time, light or electromagnetic radiation from the second dielectric waveguide 10 is still coupled directly into the first dielectric waveguide 9 in the first coupling section 23. The first receiver 24 thus receives the light or the electromagnetic radiation which is coupled into the first dielectric waveguide 9, but also the light or the electromagnetic radiation which is coupled into the third dielectric waveguide 12. The third dielectric waveguide 12 therefore requires at most one termination 22 at each of its ends.

As the two coupling sections 23 and 25 are located on opposite sides of the second dielectric waveguide 10, the third dielectric waveguide 12 must be guided via the second dielectric waveguide 10 to the side of the first dielectric waveguide 9 in order to be able to realize the common dielectric coupling section 27 there.

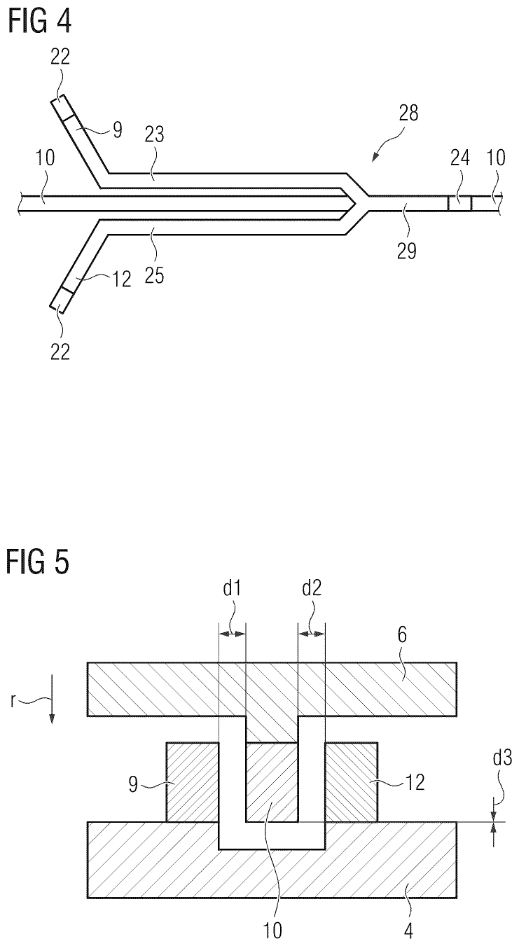

In the embodiment according to FIG. 4, likewise only a single receiver 24 is required for both dielectric waveguides 9 and 12. The configuration of the coupling arrangement essentially corresponds to that of FIG. 3. Here too, the second dielectric waveguide 10 runs between the parallel coupling sections 23 and 25 of the first and third dielectric waveguides 9 and 12. However, instead of the common dielectric coupling section 27, a dielectric combiner 28 is provided here, which combines the first dielectric waveguide 9 and the third dielectric waveguide 12 into a common waveguide 29. The first receiver 24 is arranged at the end of this common waveguide 29.

The combiner 28 brings together the first and the third dielectric waveguides 9 and 12 above the second dielectric waveguide 10 (e.g. radially outside its circular path). In this way, both the light components coupled into the first coupling section 23 and into the second coupling section 25 are combined in the common waveguide 29. The combiner 28 can be realized by 3D printing, for example, so that the waveguides are located in different planes.

The lengths and distances of the individual waveguides in the common dielectric coupling section 27 must be selected in such a manner that the maximum attenuation value is as low as possible during reception. In particular, the lengths of the coupling sections should be a multiple of the selected wavelength in the waveguide material (for example, at 60 GHz the wavelength in the waveguide material is 3 mm).

The diagrams in FIGS. 5, 6 and 7 show a cross-section, for example through one of the waveguide arrangements according to FIGS. 2 to 4. The section runs perpendicular to the image planes of these FIGS. 2 to 4. In the case of a circular gantry, the section runs in a radial direction.

FIG. 5 shows a part of the gantry 6 and a part of the base (or holding frame) 4 of a computed tomograph, for example. In relation to the circular gantry 6, the radial coordinate r in FIG. 5 runs in a downward direction. The second dielectric waveguide 10 is attached to the gantry 6. In FIG. 5, the first dielectric waveguide 9 is located to the left of the second dielectric waveguide 10 and the third waveguide 12 is located to the right of the second dielectric waveguide 10. The three waveguides 9, 10 and 12 are located here at the same height in relation to the radial coordinate r.

FIG. 5 shows a target state of the waveguide arrangement of the three waveguides 9, 10 and 12. The distance d1 between the first and second dielectric waveguides 9, 10 corresponds to the distance d2 between the second and third dielectric waveguides 10 and 12. As the waveguides are all at the same radial height, the radial distance d3 between two adjacent waveguides is always equal to 0.

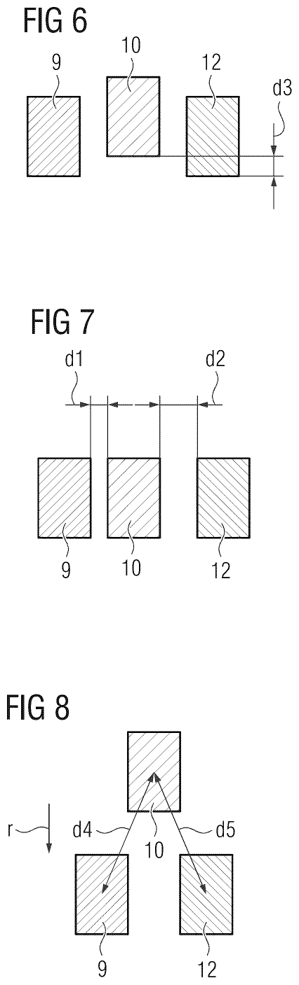

In FIGS. 6 and 7, the gantry 6 and the base 4 are not shown. Only the three waveguides 9, 10 and 12 are shown in cross-section.

FIG. 6 shows a diagrammatic view of a radial eccentricity of the gantry 6 or the second dielectric waveguide 10. The second dielectric waveguide 10 axially between the two waveguides 9 and 12 is at a certain radial distance d3 greater or less than 0 to these, at least in a rotational position. Compared to the ideal coupling arrangement of FIG. 5, the coupling strength is reduced due to this radial displacement of the second dielectric waveguide 10.

FIG. 7 diagrammatically indicates the case of an axial imbalance of the gantry 6. The second dielectric waveguide 10 is no longer located centrally between the first and the third dielectric waveguides 9 and 12. Rather, the distance d1 is smaller than the distance d2. In this case, the coupling between the second and the first dielectric waveguides 10, 9 may be greater than the coupling strength between the second and the third dielectric waveguides 9, 12 (non-linearities disregarded).

FIGS. 5 to 7 illustrate the mode of operation of the waveguide coupling. If the second dielectric waveguide 10 moves to the left or the right, the distances dl and d2 and thus also the attenuation essentially change in exactly the opposite direction. This means that there is never a very high attenuation/zero point in the coupling attenuation, which would result in an interruption of the signal transmission.

The distances and lengths of the coupler can be dimensioned in such a way that the maximum attenuation value is as low as possible during reception.

An offset of the gantry or the second dielectric waveguide 10 in a radial direction (d3) is usually less important. On the one hand, the arrangements are usually designed in such a way that fewer mechanical tolerances occur in the radial direction during gantry rotation. On the other hand, this radial offset of the dielectric waveguides also only results in a relatively small coupling variation.

FIG. 8 shows another exemplary embodiment of a waveguide arrangement in a section corresponding to FIGS. 5 to 7. The centers of the three waveguides are arranged in a triangular shape here. The centers between the first and the second dielectric waveguides 9, 10 are at a distance d4 and the centers of the second and the third dielectric waveguides 10, 12 are at the distance d5. The sum of the distances d4 +d5 is not constant when the second dielectric waveguide 10 moves relative to the first and third dielectric waveguides 9, 12. Nevertheless, even with this arrangement, a higher degree of coupling can be achieved in most movement phases than in the case of only two opposing coupling conductors. Alternatively to the arrangement in FIG. 8, the second dielectric waveguide 10 can also be arranged radially outside the other two waveguides 9, 12.

In an advantageous manner, a more robust coupling can be achieved by the coupling arrangements shown above in the exemplary embodiments, even if the movement of a waveguide is not uniform.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as a data processing facility) or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A data transmission apparatus for contactless data transmission, comprising:
a first dielectric waveguide with a first coupling section;
a second dielectric waveguide moveable opposite the first coupling section with respect to a direction of movement, the second dielectric waveguide being a first variable distance to the first coupling section; and
a third dielectric waveguide which has a second coupling section, the third dielectric waveguide being a second variable distance to the second dielectric waveguide, wherein
the first dielectric waveguide is fixed with respect to the third dielectric waveguide, and
the first coupling section and the second coupling section are of equal length along the direction of movement and lie opposite one another perpendicular to the direction of movement.

2. The data transmission apparatus of claim 1, wherein
the second dielectric waveguide is assigned to a transmitting unit of the data transmission apparatus and the first dielectric waveguide and the third dielectric waveguide are assigned to a common receiving unit of the data transmission apparatus, or
the second dielectric waveguide is assigned to a receiving unit of the data transmission apparatus and at least one of the first dielectric waveguide or the third dielectric waveguide is assigned to a transmitting unit of the data transmission apparatus.

3. The data transmission apparatus of claim 1, wherein the first coupling section and the second coupling section are symmetric with respect to one another perpendicular to the direction of movement.

4. The data transmission apparatus of claim 3, wherein the first coupling section and the second coupling section are parallel to the second dielectric waveguide.

5. The data transmission apparatus of claim 1, wherein the second dielectric waveguide extends between the first dielectric waveguide and the third dielectric waveguide.

6. The data transmission apparatus of claim 3, wherein the second dielectric waveguide is formed on a circular path with a circle center and a path radius, and the first coupling section and the second coupling section are positioned in a circumferential direction as follows:
a) one of the first coupling section and the second coupling section is positioned radially outside the circular path and the other coupling section is positioned radially inside the circular path,
b) both the first coupling section and the second coupling section are positioned with the path radius away from the circle center axially on both sides of the circular path,
c) both the first coupling section and the second coupling section are positioned radially inside the circular path and axially offset from the circular path on both sides, or
d) both the first coupling section and the second coupling section are positioned radially outside the circular path and axially offset from the circular path on both sides.

7. The data transmission apparatus of claim 1, wherein a respective receiver for detecting electromagnetic signals in the respective dielectric waveguide is arranged on the first dielectric waveguide and the third dielectric waveguide, and the data transmission apparatus is configured to combine signals from the two receivers with one another.

8. The data transmission apparatus of claim 1, wherein a single receiver is arranged on the first dielectric waveguide and the third dielectric waveguide, the single receiver is configured to receive electromagnetic signals coupled from the second dielectric waveguide into both the first dielectric waveguide and the third dielectric waveguide.

9. The data transmission apparatus of claim 8, wherein the first dielectric waveguide and the third dielectric waveguide form a second dielectric coupler in one section, with which an electromagnetic signal is transmittable from the third dielectric waveguide into the first dielectric waveguide, and the single receiver is arranged directly on the first dielectric waveguide to receive the electromagnetic signal coupled from the third dielectric waveguide into the first dielectric waveguide.

10. The data transmission apparatus of claim 9, wherein the first coupling section is a first distance from the second coupler, the second coupling section isa second distance from the second coupler, and the second distance corresponds to a total of the first distance and a multiple of a predetermined wavelength for data transmission in the first dielectric waveguide, the second dielectric waveguide, and the third dielectric waveguide.

11. The data transmission apparatus of claim 8, wherein the first dielectric waveguide and the third dielectric waveguide are guided to a dielectric combiner or form the dielectric combiner in one section with which an electromagnetic signal is conductable from both the first dielectric waveguide and the third dielectric waveguide into the single receiver.

12. An imaging apparatus comprising:
the data transmission apparatus of claim 1.

13. The imaging apparatus of claim 12, wherein the second dielectric waveguide is part of a rotatable gantry with a gantry support structure and the first dielectric waveguide and the third dielectric waveguide are part of a fixed base.

14. The imaging apparatus of claim 12, wherein the imaging apparatus is a computed tomograph apparatus or as a magnetic resonance tomograph apparatus.

15. A method for contactless data transmission, comprising:
moving a second dielectric waveguide during data transmission relative to a first coupling section of a first dielectric waveguide in a direction of movement, wherein a first distance of the second dielectric waveguide to the first coupling section varies, and wherein a second distance of a second coupling section of a third dielectric waveguide to the second dielectric waveguide varies, wherein
the first dielectric waveguide is fixed in relation to the third dielectric waveguide, and
the first coupling section and the second coupling section are of equal length in the direction of movement and lie opposite one another perpendicular to the direction of movement.

16. The data transmission apparatus of claim 4, wherein the second dielectric waveguide is formed on a circular path with a circle center and a path radius, and the first coupling section and the second coupling section are positioned in a circumferential direction as follows:
a) one of the first coupling section and the second coupling section is positioned radially outside the circular path and the other coupling section is positioned radially inside the circular path,
b) both the first coupling section and the second coupling section are positioned with the path radius away from the circle center axially on both sides of the circular path,
c) both the first coupling section and the second coupling section are positioned radially inside the circular path and axially offset from the circular path on both sides, or
d) both the first coupling section and the second coupling section are positioned radially outside the circular path and axially offset from the circular path on both sides.

17. The data transmission apparatus of claim 16, wherein a respective receiver for detecting electromagnetic signals in the respective dielectric waveguide is arranged on the first dielectric waveguide and the third dielectric waveguide, and the data transmission apparatus is configured to combine signals from the two receivers with one another.

18. The data transmission apparatus of claim 16, wherein a single receiver is arranged on the first dielectric waveguide and the third dielectric waveguide, the single receiver is configured to receive electromagnetic signals coupled from the second dielectric waveguide into both the first dielectric waveguide and the third dielectric waveguide.

19. The data transmission apparatus of claim 18, wherein the first dielectric waveguide and the third dielectric waveguide form a second dielectric coupler in one section, with which an electromagnetic signal is transmittable from the third dielectric waveguide into the first dielectric waveguide, and the single receiver is arranged directly on the first dielectric waveguide to receive the electromagnetic signal coupled from the third dielectric waveguide into the first dielectric waveguide.

20. The data transmission apparatus of claim 19, wherein the first coupling section is a first distance from the second coupler, the second coupling section isa second distance from the second coupler, and the second distance corresponds to a total of the first distance and a multiple of a predetermined wavelength for data transmission in the first dielectric waveguide, the second dielectric waveguide, and the third dielectric waveguide.
